(19) United States
(12) Patent Application Publication
(10) Pub. No.: US 2016/0220166 A1
(43) Pub. Date: Aug. 4, 2016

(54) Methodology for Discriminating Concussion Subjects from Normal Subjects by Identifying and Using QEEG correlates of concussion across 4 cognitive tasks and eyes closed condition.

(71) Applicant: Kirtley Elliott Thornton, Clover, SC (US)

(72) Inventor: Kirtley Elliott Thornton, Clover, SC (US)

(21) Appl. No.: 14/608,330

(22) Filed: Jan. 29, 2015

(51) Int. Cl. A61B 5/00; A61B 5/0484; A61B 5/04; A61B 5/048

(52) U.S. Cl. CPC A61B 5/4064; A61B 5/6803; A61B 5/048; A61B 5/04845; A61B 5/04842; A61B 5/04012

(57) ABSTRACT

Previous patents and research have focused on the problem of determining whether the quantitative EEG (QEEG) can discriminate a traumatic brain injury (TBI) subject from a normal individual. The patents and research have had varying degree of specificity in defining the variables involved in obtaining a high degree of discriminant ability. However, all research has limited its approach to the collection of eyes closed data and most confine themselves to under 32 Hertz. The present patent employs 4 cognitive activation tasks, an eyes closed task, 19 locations, the high frequency 32-64 Hz range (Spectral Correlation Coefficient (SCC) and phase algorithms) and frontal relative power of beta2 (32-64 Hz) to obtain 100% correct identification in a group of over 195 subjects (normal and traumatic brain injured (TBI)) across the 4 cognitive activation tasks and eyes closed condition. The approach is validated on a sample of 50 misclassified participants which the discriminant correctly identifies as misclassified.

### REFERENCES CITED

U.S. PATENT DOCUMENTS

| | | |
|---|---|---|
| 6,622,036 B1 | Sep. 16, 2003 | Suffin |
| 6,796,941 B2 | Sep. 28, 2004 | Williams |
| 6,985,769 B2 | Jan. 10, 2006 | Jordan |
| 7,720,530 B2 | May 18, 2010 | Causevic |
| 2009/0156954 A1 | Jun. 18, 2009 | Cox |

### OTHER PUBLICATIONS

Barr, W. B., Prichep, L. S., Chabot, R. Powell, M. R., & McCrea, M. (2012). Measuring brain electrical activity to track recovery from sport-related concussion, Brain Injury, 26(1): 8-66.
Hughes, J. R., & John, E. R. (1999). Conventional and quantitative electroencephalography in psychiatry. Journal of Neuropsychiatry and Clinical Neuroscience, 11 (2), 190-208.
Leon-Carrion, J., Martin-Rodriguez, J. F., Damas-Lopez, J., Martin, J. M., B., Dominguez-Morales, M. D. R. (2008). A QEEG index of level of functional dependence for people sustaining acquired brain injury: The Seville Independence Index (SINDI). Brain Injury, 22(1), 61-74.
Powell, J. M., Ferraro, J. V., Dikmen, S. S., Temkin, N. R., & Bell, K. R. (2008). Accuracy of mild traumatic brain injury diagnosis. Archives of Physical Medicine and Rehabilitation, 9(8), 1550-1555.
Ross, R. J., Cole, M., Thompson, J. S., & Kim, K. H. (1983). Boxers—computed tomography, EEG, and neurological evaluation. Journal of the American Medical Association, 249 (2), 211-213.
Slobounov S, Cao C, Sebastianelli W. (2009). Differential effect of first versus second concussive episodes on wavelet information quality of EEG. Clinical Neurophysiology, 120: 862-867.
Tabano, M. T., Cameroni, M., Gallozzi, G. et al. (1988). EEG Spectral analysis after minor head injury in man. Electroencephalography and Clinical Neurophysiology, 70, 185-189.
Thatcher, R. W., Biver, C., McAlaster, R., & Salazar, A. (1998). Biophysical linkage between MRI and EEG coherence in closed head injury. Neuroimage, 8 (4), 307-326.
Thatcher, R. W., Cantor, D. S., McAlaster, R., Geisler, F., & Krause, P. (1991). Comprehensive predictions of outcome in closed head injured patients: The development of prognostic equations. Annals of the New York Academy of Sciences 620, 82-101.
Thatcher, R. W., Walker, R. A., Gerson, I., & Geisler, F. H. (1989). EEG discriminant analyses of mild head trauma. Electroencephalography and Clinical Neurophysiology, 73 (2), 94-106.
Thatcher, R. W., Biver, C., McAlaster, R., & Salazar, A. (1998). Biophysical linkage between MRI and EEG coherence in closed head injury. Neuroimage, 8 (4), 307-326.
Thatcher R W, North D M, Curtin R T, et al. (2001). An EEG severity index of traumatic brain injury. J Neuropsychiatry Clinical Neurosciences, 13:77-87.
Thornton, K. (1997). The Fig Technique and the Functional Structure of Memory in Head Injured and Normal Subjects. Journal of Neurotherapy, 2 (1), 1997, 23-43.
Thornton, K. (1999). Exploratory Investigation into Mild Brain Injury and Discriminant Analysis with High Frequency Bands (32-64 Hz), Brain Injury, 477-488.
Thornton, K. (2000). Exploratory Analysis: Mild Head Injury, Discriminant Analysis with High Frequency Bands (32-64 Hz) under Attentional Activation Conditions & Does Time Heal?. Journal of Neurotherapy, 3 (3/4) 1-10.
Thornton, K. (2003). Electrophysiology of the reasons the brain damaged subject can't recall what they hear. Archives of Clinical Neuropsychology, 17, 1-17.
Thornton, K & Carmody, D. (2009b). Integrative Clinical Psychology, Psychiatry and Behavioral Medicine: Perspectives, Practices and Research Thornton, K. & Carmody, D. Chapter Title: Traumatic Brain Injury and the Role of the Quantitative EEG in the assessment and remediation of cognitive sequelae, December 2009. 463-508.
Thornton, K. (2014) Chapter Title: The Role of the quantitative EEG in the diagnosis and rehabilitation of the traumatic brain injured patient, Concussions in Athletics: From Brain to Behavior, Chapter 20, Eds. Semyon, M. Slobounov and Wayne Sebastianelli, Springer publ., 2014, NY, N.Y., pgs. 45-362.
Thornton, K. (2014). A QEEG activation methodology which obtains 100% accuracy in the discrimination of traumatic brain injured from normal and does the learning disabled show the brain injury pattern?, Neuroregulation, 1 (3-4), 209-217.
Trudeau, D. L., Anderson, J., Hansen, L. M., Shagalov, D. N., Schmoller, J., Nugent, S., et al. (1998). Findings of mild traumatic brain injury in combat veterans with PTSD and a history of blast concussion. Journal of Neuropsychiatry and Clinical Neurosciences, 10 (3), 308-313.
Tysvaer, A. T., Storli, O. V., Bachen, N. I. (1989). Soccer injuries to the brain. A neurologic and electroencephalographic study of former players, Acta Neurologica Scandinavica, Vol. 80 (2). 151-156.

### BACKGROUND

Previous utility patents addressing the discriminate ability of the quantitative EEG to differentiate traumatic brain injury from normal groups have typically relied upon one session of eyes closed data and a frequency range of 0 to 32 Hertz. To assess communication patterns (SCC and phase) the different hardware manufacturers and software engineers have employed different mathematical algorithms to calculate these values. The Lexicor SCC variable assesses the degree of similarity across a period of time (epoch) while the phase variable measures the time delay of a frequency between two locations.

A patent search in this area revealed patents which focus on this general area without specifically attempting to differentiate TBI from normal. These patents generally indicate that their approach will be able to differentiate brain injured groups from normals without providing specific information on exactly what variables will be employed. In addition, the patents do not typically report discriminant results in terms of false positives and false negatives.

The value of a patent which can quickly and reliably differentiate between a TBI subject and a normal subject would be an important contribution to the sports arena, emergency rooms, and returning Iraq and Afghanistan war veterans, as TBI has been considered the signature wound of these wars.

### Previous Patents

The Suffin patent (U.S. Pat. No. 6,622,036 B1) addressed focused on gathering QEEG data for “classifying, diagnosing and treating physiologic brain imbalances”. The patent's methodology is to compare a subject's QEEG response to a clinically identified comparison group or normal group to determine the brain's imbalances, examines the differences for possible intervention decisions, and examine the QEEG response to different medication interventions.

The QEEG variables under consideration were the absolute magnitudes of the different frequencies (0 to 35 Hz), relative power, coherences, peak frequency and symmetry measures. They list a number of clinical conditions that they have in their database, including traumatic brain injured. However, they did not discuss the parameters or statistical method that would be involved in differentiating normal subjects from traumatic brain injured subjects. They also did not report any discriminant analysis results of TBI vs. normal.

The Williams patent (U.S. Pat. No. 6,796,941 B2) relates to “data evaluation equipment and procedures for the monitoring and management of brain injuries in mammals”. The EEG is just one of the measures proposed. The patent does not discuss specific EEG parameters which relate to brain injury, but focused on seizure activity in terms of the EEG.

The Jordan patent (U.S. Pat. No. 6,985,769 B2) proposed a “method and system for automated real time interpretation of brain waves in an acute brain injury of a patient using correlations between brain wave frequency power ratio and wave morphology, determine by a measure of the rhythmicity and variability of the brain wave as a function of the slope of the brain wave upstroke, the arc of the brain wave, and the synchronicity of the brain wave”. The authors propose that a power ratio such as alpha-beta/theta-delta would be the useful variable. On the basis of the data they have collected they have argued that the brain injury results in an increase in the slower frequencies, compared to a normal referenced group. They do not, however, discuss the issue of coherence or phase nor provide discriminant analysis.

The Causevic patent (U.S. Pat. No. 7,720,530 B2) addresses a field-deployable concussion detector. They propose using less than 10 electrodes and extending the frequency range to 50 Hz and even 1000 Hz. The patent proposes to employ absolute power, relative power, symmetry and coherence as the critical differentiating variables between normals and the traumatic brain injured (TBI). However, they never provide what specific variables are relevant to the TBI discriminant. Thus, the patent is a method to discover what variables are relevant to the TBI situation.

The Cox patent (No. 2009/0156954 A1) addresses diagnosing attentional impairment using EEG data and include the traumatic brain injured subject as having as having attentional problems. The patent mostly discusses the ADD/ADHD diagnosis issue in terms of elevation of the lower frequencies (theta, in particular).

### PREVIOUS RESEARCH ON DISCRIMINATING TBIS FROM NORMAL WITH THE QEEG

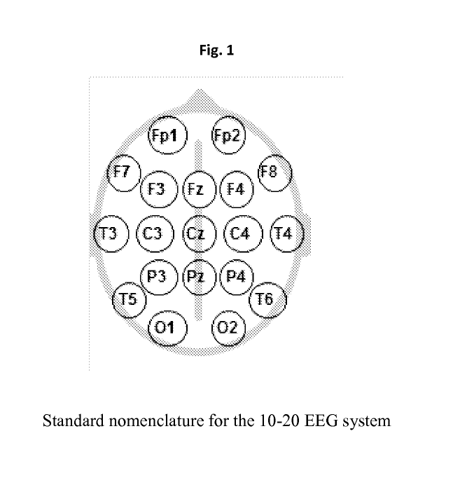

FIG. 1 presents the locations and nomenclature for the standard 10-20 system which is employed in the quantitative EEG field. The research involved all locations.

FIG. 1—Locations in the 10-20 EEG system

Insert FIG. 1

### QEEG Variable Concepts

The concept of coherence employs the Lexicor's algorithm of Spectral Correlation Coefficients and Lexicor's Phase algorithm. Different hardware/software manufacturers employ different algorithms to calculate these values. The following measures were available for the analysis. The following text describes the QEEG variables involved in the analysis.

Arousal Measures

Relative Magnitude: the relative EEG magnitude within a frequency band (absolute magnitude in a particular band divided by the total microvolts generated at a particular location in all bands)

Connectivity Measures

Coherence: the average similarity between the waveform morphology in a particular frequency band from two locations over an epoch (a one-second period of time in this research). The measure has been conceptualized as the strength/number of connections between the two locations. Lexicor software provides an amplitude matching algorithm. However, an alternate conceptualization of the relations (SCC and phase) could refer to the quality of signal transmission, with degradation of the signal reflected in lower values.

Phase: the time lag between waves from two locations in a particular band as defined by how soon after the beginning of an epoch a particular waveform at location #1 is matched in location #2 (amplitude)

### Frequency Ranges

The frequency range employed is 0-64 Hertz. The 5 frequency bands are defined as follows:

| | | |
|---|---|---|
| Delta: 0-4 Hz | Theta: 4-8 Hz | Alpha: 8-13 Hz |
| Beta1: 13-32 Hz | Beta2: 32-64 Hz | |

Previous research which has addressed the issue of statistically discriminating traumatic brain injury subjects from normal individuals include publications by Thatcher (1989, 1998), Hughes & John (1989), Tabano et al. (1988), Trudeau et al., (1998), Bar et al. (2012), Leon-Carrion et al. (2008), and Thornton (1997, 1999, 2000, 2003).

Tabano, Cameroni and Gallozzi (1988) investigated posterior activity of subjects (N=18) at 3 & 10 days following a MTBI and found an increase in the mean power of the lower alpha range (8-10 Hz) and reduction in fast alpha (10.5-13.5 Hz) with an accompanying shift of the mean power of the lower alpha range (8-10 Hz) and reduction in fast alpha (10.5-13.5 Hz) with an accompanying shift of the mean alpha frequency to lower values. They also reported a reduction in fast beta (20.5-36 Hz) activity. They did not conduct a discriminant analysis of TBI vs. normals.

Thatcher, Walker, Gerson, & Geisler (1989) article was the first to attempt to conduct a discriminant function analysis. They used the eyes closed QEEG data to differentiate between 608 MTBI adult patients and 108 age-matched controls and obtained a discriminant accuracy rate of 90%. Moderate to severe cases were not included in the analysis, nor was the high frequency gamma band (32-64 Hz) or cognitive activation conditions. The useful QEEG measures included increased frontal theta coherence (Fp1-F3), decreased frontal beta (13-22 Hz) phase (Fp2-F4, F3-F4), increased coherence beta (T3-T5, C3-P3), and reduced posterior relative power alpha (P3, P4, T5, T6, O1, O2, T4). Three independent cross validations (reported within the original research) resulted in accuracy rates of 84%, 93%, and 90%.

Thatcher, Biver, McAlaster, Camacho and Salazar (1998) were able to demonstrate a relationship between increased theta amplitudes and increased white matter T2 Magnetic Resonance Imaging (MRI) relaxation times (indicator of dysfunction) in a sample of mild TBI subjects. Decreased alpha and beta amplitudes were associated with lengthened gray matter T2 MRI relaxation times. The subjects were 10 days to 11 years post injury. This study integrated MRI, QEEG (eyes closed) and neuropsychological measures in a sample of MTBI subjects.

Thatcher (2001) employed this method to develop a severity of brain injury value.

One review of the research in the traumatic brain injury area indicated that numerous eyes-closed EEG and QEEG studies of severe head injury (Glascow Coma Scale (GCS) score of 4-8) and moderate injury (GCS score of 9-12) have agreed that increased theta and decreased alpha power (microvolts) and/or decreased coherence and symmetry deviations from normal groups often characterize such patients (J. R. Hughes & John, 1999).

The authors asserted that changes in these measures provide the best predictors of long term outcome. The Thatcher discriminant function (Thatcher et al., 1989) correctly identified 88% of the soldiers with a blast injury history and 75% with no blast injury history (Trudeau et al., 1998).

Other studies have reported that similar QEEG abnormalities are correlated with the numbers of bouts or knockouts in boxers (Ross, Cole, Thompson, & Kim, 1983) and with professional soccer players who frequently used their heads to affect the soccer ball's trajectory (“headers”; Tysvaer, Storli, & Bachen, 1989). Neither of these research reports attempted to develop a discriminant function analysis.

Barr et al. (2012) took EEG recordings from 5 frontal locations (F7, Fp1, Fp2, F8 and a location below Fz) immediately post-concussion, and 8 and 45 days after. They examined the frequency range up to 45 Hertz on measures of absolute power, relative power, mean frequency, coherence, symmetry and a fractal measure. Using a brain injury algorithm, abnormal features of brain electrical activity were detected in athletes with concussion at the time of injury which persisted beyond the point of recovery on clinical measures.

Features that contributed most to the discriminant applied in this study included: relative power increase in slow waves (delta and theta frequency bands) in frontal, relative power decreases in alpha 1 and alpha 2 in frontal regions, power asymmetries in theta and total power between lateral and midline frontal regions, incoherence in slow waves between fronto-polar regions, decrease in mean frequency of the total spectrum composited across frontal regions and abnormalities in other measures of connectivity (including mutual information and entropy). A resulting discriminant score was employed to distinguish between the TBI and normal group. If the discriminant score was above 65 there was a 95% probability that the individual had experienced a TBI. The average discriminant score change from the immediate post-concussion score of 75 to a score of 55 some 45 days later, thus rendering its ability to discriminant after the original concussion not as useful as would be desired.

The TBI's cognitive status, as assessed with neuropsychological measures, had returned to the “normal” range at day 45, although brain abnormalities were still present (TBI sample size=59). The researchers did not internally attempt to replicate the findings within the sample that they had obtained.

Leon-Carrion, Martin-Rodriguez, Damas-Lopez, Martin & Dominguez-Morales (2008) documented the discriminant ability of the QEEG to accurately classify brain injury in 100% of the “training set sample” (N=48) and obtained a 75% correct classification in “an external cross-validation sample” of 33. The average time between the QEEG evaluation and incident (TBI, CVA) was 22 months. The authors noted that “coherence measures were the most numerous variables in the function”, employing the frequency range of 1-30 Hz.

Previous research by Thornton (1997, 1999, 2000) focused on the damage to the Spectral Coherence Correlation Coefficients (SCC—based upon the Lexicor algorithms) and phase values in the beta2 (gamma; 32-64 Hertz) range when comparing the traumatic brain injured subject to the normal group during eyes closed and different cognitive activation tasks. The TBI sample size ranged from 22 to 32 with 52 normal phase in the 1999 & 2000 studies. Lexicor Medical Technology (Boulder, Co.) company developed their own algorithms for coherence and phase. The coherence measure algorithms were not the same as employed in the Barr et al. (2012) study.

The Thornton results (1997, 1999, 2000) did not indicate any deficits in the amplitudes or relative power of delta, theta or alpha. In the Thornton (2003) article (addressing auditory memory) the alpha level was set to 0.02 due to high number of significant findings in the beta2 SCC and phase values predominantly in the values involving the frontal lobe. The TBI group showed lower beta2 coherence (SCC) values. The article studied the relations between the QEEG variables and memory performance in 85 TBI patients and 56 normal subjects.

The claim of this patent is to that it is possible obtain 100% discriminant accuracy across 5 cognitive tasks. Confirmatory evidence is obtained by employing a misclassification (of both normal and brain injured participants) approach and testing the ability of the discriminant analysis to correctly identify the misclassification across the 5 tasks. The discriminant analysis was successful in 100% of the 50 misclassifications involving the cognitive and eyes closed tasks, as reported in the Thornton (2014) publication.

Almost all of the previous research has not examined the beta2 frequency in terms of absolute, relative power or phase and coherence (SCC) relations. The data available to the author was reexamined for potentially useful variables. The standard eyes closed task collects data for 300 seconds. The Auditory Attention task requires the eyes closed subject to place their hand on their right knee and raise their index finger whenever they hear the sound of the pen tapping on a table.

The Visual Attention task has the subject look at a laminated sheet of upside down Spanish text. Similar to the Auditory Attention task, the subject has their right hand on their right leg. When they see the flash of a laser light on the sheet of paper they are to raise their index finger. Each of the attention tasks last 200 seconds each. The reading task requires the subject to silently read a story presented on a laminated sheet of paper for 100 seconds. Thus the evaluation requires, at present, 800 seconds or 13.3 minutes. Reliability data for QEEG data typically is in the 0.90 to 0.95 range.

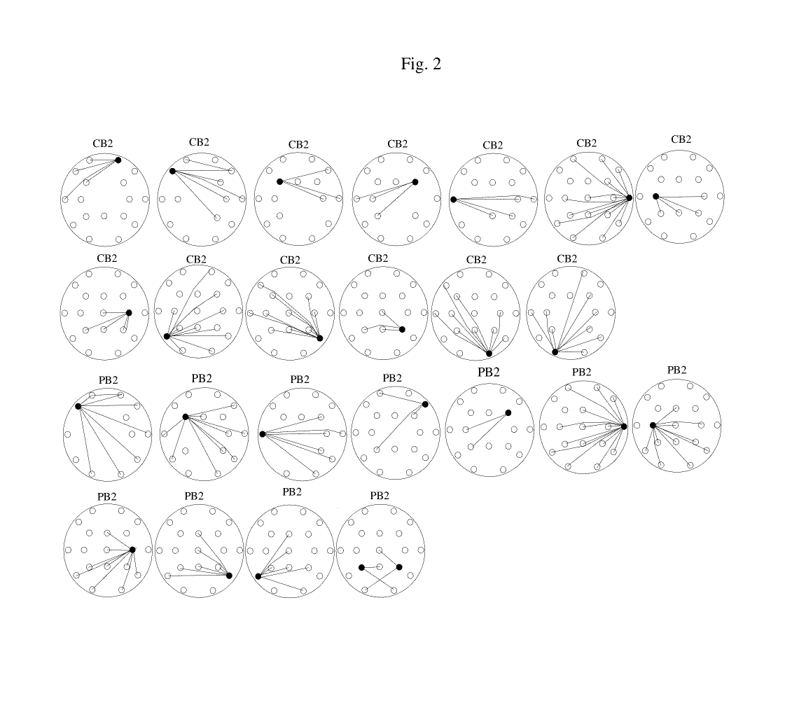

The discriminant analysis employed all 19 locations (FIG. 1) and the SCC and phase values (32-64 Hz) of all the interrelations between these 19 locations and the relative power of beta2 (32-64 Hz) values from 6 frontal locations (Fp1, Fp2, F7, F8, F3, F4). FIG. 2 presents the relations which were significantly below the normative reference group (alpha set to 0.05) for the SCC and phase values. The concussed individual averaged about 0.50 standard deviations (SD) below the normative group on the phase and coherence values and were about 0.50 SD above the normative reference group on the frontal relative power of beta2 values.

FIG. 2 presents the significant SCC and phase deficits in the TBI participant.

FIG. 2—Significant SCC and phase deficits in the TBI participant

Insert FIG. 2
CB2=Coherence (SCC) beta2: PB2=Phase beta2

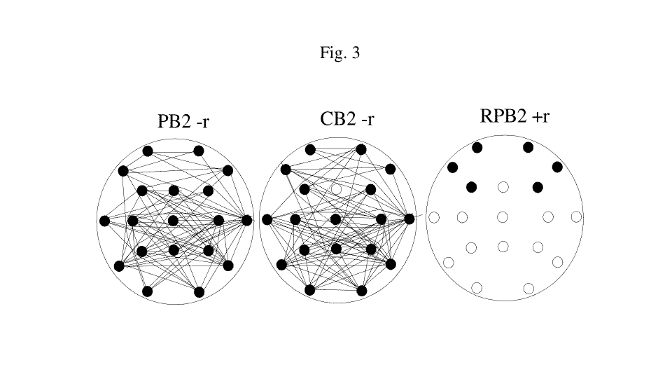

FIG. 3 presents a summary figure of the significant SCC and phase deficits in the TBI participant.

FIG. 3—Summary head figures

Insert FIG. 3

CB2=Coherence (SCC) beta2: PB2=Phase beta2; RPB2=Relative Power Beta2

The following tables present the discriminant function (General Discrimination analysis Model employed in Statistica—vs. 8) results for the different tasks. All of the tables indicate 100% accuracy in discriminating normal from brain injury. The time between the date of the head injury and evaluation ranged from 12 days to 30 years. The average age of the total sample (listening task data) was 39.47 with a range between 14.08 years to 72.42 years. There were 95 males and 102 females in the listening task group (total N=197). There were 88 participants classified as TBI and 109 participants classified as normal. There was a range of 162-197 subjects involved in the different conditions. Tables 1-5 present the resulting discriminant analysis for the five tasks. As the tables indicate the discriminant analysis were 100% effective in distinguishing between the TBI and normal participants.

TABLE 1
Classification Matrix (EC)—Eyes Closed

| | EC | TBI | N |
|---|---|---|---|
| | Correct | P = .56 | P = .44 |
| TBI | 100 | 102 | 0 |
| N | 100 | 0 | 81 |
| Total | 100 | 100 | 81 |

TABLE 2
Classification Matrix (AA)—Auditory Attention

| | AA | TBI | N |
|---|---|---|---|
| | Correct | P = .51 | P = .49 |
| TBI | 100 | 90 | 0 |
| N | 100 | 0 | 86 |
| Total | 100 | 90 | 86 |

TABLE 3
Classification Matrix (VA)—Visual Attention

| | VA | TBI | N |
|---|---|---|---|
| | Correct | P = .52 | P = .48 |
| TBI | 100 | 87 | 0 |
| N | 100 | 0 | 81 |
| Total | 100 | 87 | 81 |

TABLE 4
Classification Matrix (Listen) - Auditory Memory

| | VA | TBI | N |
|---|---|---|---|
| | Correct | P = .45 | P = .55 |
| TBI | 100 | 88 | 0 |
| N | 100 | 0 | 109 |
| Total | 100 | 88 | 109 |

TABLE 5
Classification Matrix (RS) - Reading

| | VA | TBI | N |
|---|---|---|---|
| | Correct | P = .46 | P = .54 |
| TBI | 100 | 75 | 0 |
| N | 100 | 0 | 87 |
| Total | 100 | 75 | 87 |

To determine if the discriminant algorithm could accurately indicate a misclassification, five TBI subjects and five normal subjects were misclassified (for each task) as to their status and the discriminant analysis was recalculated to determine if the inaccurate classification was identified. Ten different subjects were selected for each task for a total of 50 misclassifications. Table 6 presents the results of this analysis.

Row 1 indicates the # of errors in the initial discriminant analysis. The 0 number indicates no misclassifications. Row 2 indicates how the group of 5 participants were misclassified for each task. The label MC as TBI indicates that 5 normal participants were misclassified as TBI. The MC as N label indicates that 5 TBI participants were misclassified as normal. Row 3 indicates the number of errors resulting for the group in the b column. For the 5 TBI participants misclassified as normal the reanalysis indicated the misclassification and thus 0 errors. Row 4 indicates the error rate for the normal participants who were misclassified as TBI. Row 5 indicates the overall error rate across both methods. As the table indicates there were no errors in any of the approaches. Table 6 presents the classification error values.

TABLE 6
Classification Error Values

| | | a)MC | b)MC | | c)MC | d)MC | | e)MC | f)MC | | g)MC | h)MC | | i)MC | j)MC |
|---|---|---|---|---|---|---|---|---|---|---|---|---|---|---|---|
| | EC | as TBI | as N | AA | as TBI | as N | VA | as TBI | as N | LS | as TBI | as N | RS | as TBI | as N |
| 1)Initial | | 0 | 0 | | 0 | 0 | | 0 | 0 | | 0 | 0 | | 0 | 0 |
| Analysis | | | | | | | | | | | | | | | |
| 2)MC | | MC as | MC as | | MC as | MC as | | MC as | MC as | | MC as | MC as | | MC as | MC as |
| | | TBI | N | | TBI | N | | TBI | N | | TBI | N | | TBI | N |
| 3)MTBI | | | 0 | | | 0 | | | 0 | | | 0 | | | 0 |
| 4)MN | | 0 | | | 0 | | | 0 | | | 0 | | | 0 | |
| 5)# Errors | | 0 | 0 | | 0 | 0 | | 0 | 0 | | 0 | 0 | | 0 | 0 |

MC = Misclassified:
EC: Eyes Closed:
AA: Auditory Attention:
VA: visual attention:
LS: listening to paragraph:
RS: reading
Initial Analysis: indicates the preliminary discriminant analysis results
Misclassification: indicates the results of misclassifying the subjects and the results of discriminant reanalysis to determine if algorithm can discern misclassification, number indicates the subject # that was not accurately identified;
0 indicates 100% correct identification of misclassified subject
MTBI: TBI subjects misclassified as normal
MN: Normal subjects misclassified as TBI

The problem of determining if a person in a sports event has experienced a concussion presents two additional difficulties. The first is whether the initial post concussive brain state is going to be significantly different that the concussed brain state some 12 days to 30 years later. The second is that a previous concussion could be affecting the results. It is therefore possible that the discriminant approach will be identifying the previous concussion and there is not a concussive event presently.

Evidence towards the first problem is provided in the Thornton (2000) and Thatcher (1998) articles. These studies indicate that the EEG concussed pattern does not change over time. Thus, the concussed brain injury QEEG signature should be evident at the time of the initial concussion.

In addition, the work of Barr et al. (2012) indicates that the TBI's brain pattern remains affected despite improvement in cognitive function, thus indicating a compensation response, i.e. the brain employs other resources to accomplish the cognitive task. The compensated QEEG response pattern was also documented in the Thornton (2003) article and book chapter (Thornton & Carmody, 2009) showing a right hemisphere compensation approach.

To address the second problem, the work of Slobounov, Cao & Sebastianelli (1990) addressed the problem of the second concussion. The researchers employed a wavelet entropy (EEG-IQ) algorithm. The algorithm addresses the Information Quality of EEG (EEG-IQ1) which first applies discrete wavelet transform (DWT) to the EEG signal and then calculates the traditional Shannon Entropy of the wavelet coefficients. This measure was reduced at temporal, parietal and occipital locations after the first concussion and particularly after the second.

In addition, Slobounov et al. (1990) reported that the EEG-IQ measure was affected more after the second concussion compared to the first concussion. Thus, the second concussion showed a similar EEG effect. The QEEG pattern was also slower to recover. In addition, the shorter the time interval between the two concussions resulted in larger reductions of EEG-IQ values. The results also indicated a better outcome after the first compared to the second concussion. Thus the authors were able to show that a similar effect occurred during the second concussion and it was more pronounced. Therefore, it is logical to assume a similar effect would occur for the variables that this patent employs.

On the procedural level, the hypothetically concussed individual's data could be entered into the Statistica spreadsheet or equivalent software (containing the presently available data) and the five discriminants run to determine if the subject had experienced a concussion during an athletic event or other trauma. This approach assumes there is no previous concussion. All five tasks or combination could be administered to ensure accuracy. If a set of data on one task is contaminated by artifact it wouldn't be employed for the decision. As all the tasks have a high degree of discriminability there would be no loss of discriminative power.

To address the problem of a previous concussion the following methodology could be employed. A baseline functioning on the five tasks could be obtained. As the QEEG variables are not subject to conscious manipulation, the probability that the athlete would feign a bad baseline response so that the on-field evaluation would be employing an “impaired” baseline would be eliminated. The baseline evaluation would collect cognitive performance as well as the QEEG data. The procedure could be employed to determine if the participant has a brain injury pattern from a previous concussion, employing the algorithms developed in the initial research reported here. The main value of the baseline evaluation is to provide data for comparison to the evaluation which takes place during the subsequent athletic event.

To determine the presence of concussion during a sports event the participant would undergo the same evaluation as occurred in the baseline evaluation for the comparison analysis and the following analysis conducted to determine if a new brain injury has occurred.

Eighty-six of the 171 coherence variables showed a significant decrease (alpha @ 0.05) (compared to the normative group) for an average change of 0.47 SD in the direction of impaired QEEG variables. Seventy-seven of the 171 phase beta2 variables showed a significant difference from the normative group with the average SD difference of 0.44. However, many of the variables were close to the 0.05 level. The coherence and phase beta2 variables and frontal relative power of beta2 values indicated by the initial research would be employed in the analysis. A 0.50 SD (coherence values) and 0.50 SD (phase values) change value (from baseline assessment) would be employed as the conservative cut off values to render the decision. The relative power of beta2 values for the TBI group were 0.47 SD above the normative group. Thus employment of a 0.50 SD average increase (from baseline assessment) for the 6 frontal locations would be a conservative value for the cutoff. The medical personnel involved in the decision could employ the logic and data of this research as well as the knowledge of the event to determine the presence of a brain injury.

If the discriminants indicated a concussion, then the player would be taken out of the game and his progress assessed in the days/weeks following the concussion.

### BRIEF SUMMARY

The claim of this patent is that by employing the Spectral Correlation Coefficients (SCC) and phase relations and 6 frontal relative power of beta2 values obtained during an activation QEEG evaluation involving 5 tasks (eyes closed, auditory and visual attention, auditory memory and reading silently) a 100% correct identification of the traumatic brain injured group and normal group with no false positives or false negatives can be obtained. As the claim indicates that the results can be obtained across of number of cognitive tasks, the claim is applicable to any cognitive task which could be employed. The patent is not claiming an EEG signal, but rather the value of that signal in reference to a normative database (and the subject's own baseline data) in determining if a TBI has occurred.

Thus the method can be useful in the immediate diagnosis of a TBI (as in sports events). The claim of this patent is particularly relevant to a) the sports concussion area; b) emergency room diagnosis of the traumatic brain injury (TBI) patient, as approximately 56% of TBIs are missed in the emergency room (Powell et al., 2008) by present diagnostic approaches (rating scales, behavioral observations); c) soldiers in combat situations; d) and returning military veterans who may have experienced a TBI during combat.

1. A method for diagnosing with 100% accuracy whether a concussion has occurred by engaging a subject in a diagnostic test of the human brain to ascertain a subject's values on specific quantitative EEG (QEEG) variables; by attaching an electro-cap on the head, measuring the spectral correlation coefficients (SCC) and phase relations between the 19 locations in the 32-64 Hz frequency range, measuring the relative power of the 32-64 Hz frequency range in 6 frontal locations (Fp1, Fp2, F7, F8, F3, F4) during the following 5 tasks; eyes closed, auditory attention, visual attention, listening to stories and reading, recording the QEEG values and memory scores; converting the subject's QEEG data obtained during the recording into an ASCII file; importing the QEEG data into a statistical computer analysis program loaded on a computer; examining the subject's QEEG data during each of the 5 tasks in relation to a normative database on the SCC and phase values (32-64 Hz) and frontal 32-64 Hz relative power values to determine if these values match the previous pattern of a concussion; by employing the previously developed 5 discriminant algorithms (on each of the 5 tasks separately) to determine if the algorithm indicates that the subject is classified as experienced a concussion or has not experienced a concussion by the 5 algorithms, which indicate lower (than the normative reference group) SCC and phase values and elevated (compared to the normative reference group) frontal relative power of the 32-64 Hz range for the concussed subjects.

2. A method for diagnosing with 100% accuracy whether a concussion has occurred in a recent possible concussive event (e.g. sports concussion) by engaging a subject in a baseline diagnostic test of the human brain prior the recent possible concussive event to ascertain a baseline of the subject's values on specific quantitative EEG (QEEG) variables; by attaching an electro-cap on the head, measuring the spectral correlation coefficients (SCC) and phase relations between the 19 locations in the 32-64 Hz frequency range, measuring the relative power of the 32-64 Hz frequency range in 6 frontal locations (Fp1, Fp2, F7, F8, F3, F4) during the following 5 tasks; eyes closed, auditory attention, visual attention, listening to stories and reading, recording the QEEG values and memory scores; converting the subject's QEEG data obtained during the recording into an ASCII file; importing the QEEG data into a statistical computer analysis program loaded on a computer for analysis in the event of a possible future concussion; when the subject is thought to have undergone a subsequent concussion the subject is re-examined on the same 5 tasks, employing the same variables and frequency range employed in the baseline task, the QEEG data is converted to an ASCII file and imported into a statistical computer analysis program, examining the subject's QEEG data during each of the 5 tasks in relation to the subject's previous baseline values on the SCC and phase values (32-64 Hz) and frontal 32-64 Hz relative power values to determine if these values match the previous baseline values or evident values which are 0.50 standard deviations (SD) below the subject's baseline values on the SCC and phase values and 0.50 SD above previous baseline values in terms of frontal relative power of the 32-64 Hz range on the variables which differentiated concussed subjects from normals in the original sample; the diagnosis of a concussion is recommended if the SCC and phase values are 0.50 SD below the baseline value and 0.50 SD above the baseline on the relative power for the 32-64 Hz value, for the locations reported in this application.
